(19) United States
(12) Patent Application Publication
(10) Pub. No.: US 2022/0349875 A1
(43) Pub. Date: Nov. 3, 2022

(54) METHOD TO INDEPENDENTLY ANALYZE MULTIPLE BIOLOGICAL PROCESSES IN ENCAPSULATED 3D CELL CO-CULTURES

(71) Applicant: UNIVERSITÉ DE GENÈVE, Genève (CH)

(72) Inventors: Grégory SEGALA, Saint-Nauphary (FR); David PEJOSKI, Cointrin (CH); Aurélien ROUX, Pully (CH); Didier PICARD, Plan-les-Ouates (CH); Dimitri Vincent MOREAU, Lajoux (FR); Bryan Josué BOURRAT, Prévessin-Moëns (FR)

(73) Assignee: UNIVERSITÉ DE GENÈVE, Genève (CH)

(21) Appl. No.: 17/763,561

(22) PCT Filed: Sep. 23, 2020
(86) PCT No.: PCT/EP2020/076559

§ 371 Date: Mar. 24, 2022

(30) Foreign Application Priority Data
Sep. 24, 2019 (EP) 19199296.5

(51) Int. Cl. G01N 33/50; C12Q 1/6897

(52) U.S. Cl. CPC G01N 33/5011; C12Q 1/6897; C12Q 1/66

(57) ABSTRACT

A multiplexed and encapsulated 3D cell co-culture drug testing or screening method which discloses an in vitro drug testing kit suitable for testing the effect of one or more drugs of interest on multiple biological processes in one or more target cell types.

### FIELD OF THE INVENTION

The invention relates to a multiplexed and encapsulated 3D cell co-culture drug testing or screening method. The invention also discloses an in vitro drug testing kit suitable for testing the effect of one or more drugs of interest on multiple biological processes in one or more target cell types.

### BACKGROUND OF THE INVENTION

In vitro screening or testing of molecular entities on biological cells constitutes an essential procedure that is used for the discovery of new pharmaceuticals, and also more recently, during ‘precision medicine’ testing to define the best drug(s) to treat patients. In vitro molecule screening can be used at both the early stages of drug discovery to identify ‘hit’ molecules, or at subsequent stages to define a set of ‘lead’ entities from the hits, which are defined as hits that have multiple favourable characteristics. Because of the large numbers of chemical entities that are screened, in vitro drug testing can be very costly in terms of time and resources. Despite these large investments, in vitro drug testing is not always successful in defining good candidates with the desired biological effects for a number of reasons, mainly the poor clinical relevance of the cell cultures used. Typical monolayer cell culture used in drug screening do not reflect the 3D tissue physiology, cell subset diversity, or multi-organ interactions that occur in vivo. Additionally, drug screening has usually been conducted using a single experimental readout per test, or two readouts at most. Thus, multiple rounds of screening that focus on different properties of the molecule are required to identify drugs with the best overall drug development profile. For example, drug screening usually commences with an evaluation of biological activity on target cells and is then followed by additional screening rounds to identify hepatotoxicity or neurotoxicity, including of metabolites that are only generated in the presence of hepatocytes. Moreover, important physiological interactions exist in vivo, for example the metabolization of a prodrug like tamoxifen by the liver for its activation in another tissue, the breast for tamoxifen. Notably, hepatocytes are often absent from the original drug screen, or if present, they may be in direct contact with other cell types, which is does not reflect in vivo cell biology. To not include these important physiological interactions between cell types could lead to miss therapeutics that would be efficient in vivo. Therefore, a problem in the current drug screening landscape, which results in the need for a multiplicity of tests to evaluate different properties and effects of potential drug candidates, is the lack of methods that can simultaneously address all of the abovementioned phenomena of cell culture drug testing. Specifically, improved drug screening or testing methods would have the ability to multiplex the evaluation process and make use of cell culture systems that are more physiologically relevant.

A distinction can be made between target-based and phenotypic drug screening. A target-based drug screening approach relies on prior identification of a specific and often unique molecular pathway, receptor, or other biomolecule that can be targeted to achieve the desired biological effects on cells of interest. For example, the estrogen receptor on (neoplastic) breast-derived cells has been demonstrated to be a viable target for endocrine therapy. Experimental cell lines can be genetically engineered to express an estrogen-dependent reporter to make the effects of drugs on this receptor easier to detect or study in vitro. In contrast, phenotypic screening does not rely on prior knowledge of exact molecular pathways nor cellular targets that a drug should act upon to achieve the desired biological effects on the cells of interest. In phenotypic screening, generalized biological processes such as cell death, cell proliferation, cell toxicity, and immune cell activation can be detected using well-established genetic or protein markers for these general phenotypic changes. The exact molecular partner that is targeted by the drugs identified using phenotyptic screening remains unknown until additional test, which are beyond the scope of this patent, are performed.

Previous attempts to improve in vitro drug screening and testing have only partially addressed the aforementioned problems, and to the best of Applicant's knowledge have never addressed all of the aforementioned problems simultaneously. For example, EP 1 235 935 B1 (Rosetta Inpharmatics LLC), describes the use of ‘multiplexed’ (multiple per cell) gene reporter cell culture systems, to identify drugs that inhibit the function of target genes which are related to the fitness of a cell i.e. target-based drug screening. Multiple genes can be detected after the co-culture of different types of cells, however the teaching of that document does not extend to 3D spheroid cell cultures, and the ability to independently analyze the different types of co-cultured cells is not possible.

Multiplexed phenotypic drug testing has also been previously published (see PMID 20116850), demonstrating the possibility to investigate drug-induced modulation of a large panel of proteins in cell supernatants. Once again, this drug testing methodology does not encompass the use of 3D cell cultures such as spheroids to increase the physiological relevance of the system. Furthermore, is not possible to detect in which cells the biological processes have been modulated because the assay readout comprises of soluble protein(s).

There is an increasing body of previous art and academic publications related to cell culture systems with improved physiological relevance, notably 3D spheroid cultures. For example, EP 2 491 386 B1 (Plasticell Ltd), describes cell culture in a plurality of microcapsules that are each labeled with two or more labels, either directly within the capsule material or inside the capsule interior. This document also includes ‘split-pool cell’ or ‘combinatorial cell’ cultures, that mainly aim to define how cells of the same type, but of varying differentiation statuses, may have different phenotypic or genotypic properties from one another, e.g. differential proliferation capacities, or expression of particular genes or proteins. Similarly, KR 101726063 B1 (UNIV SUNGKYUNKWAN RES & BUS [KR]), describes the labeling of liposomes (capsule like lipid bilayers), with a variety of fluorochromes, which is used to encapsulate 3D tumor cell cultures. While these documents are similar to the current art in that different fluorochomes (quantum dots) can be used to label the capsules, neither of them disclose any means of detecting multiple biological processes per test, nor easily discriminating the various capsules by microscopy because the liposomes are mainly used for genetic diagnosis of tumors.

### BRIEF DESCRIPTION OF THE INVENTION

The invention aims at improving in vitro drug testing i.e. screening of new or previously approved chemical or biological entities to identify medical treatments with the desired activity in at least two types of target cells, with the least adverse side effects. These improvements include (1) increasing the translational relevance, via the use of multiple cell types co-cultures that better represent a multi-tissue organism, (2) increasing the information quantity and quality that can be obtained from the test, by increasing the number of biological processes or readouts as well as the number of different cell types that can be analysed independently in the test, and (3) increasing drug testing efficacy, by reducing the time, costs, and the amount of testing that is required to be performed in vivo, which are all relevant attributes during the shortlisting of better drug candidates. The present invention allows for an improved selection of drug candidates, to progress with better reliability to subsequent in vivo testing stages as a result of the features described in points (1) and (2).

One of the objects of the present invention is to provide an in vitro method suitable for drug testing that is capable of analyzing multiple biological processes in one or more cell types, the method comprising:
a) expressing in each of said one or more cell types to be analyzed in the same in vitro drug testing assay, at least one fluorescent or bioluminescent reporter gene for each biological process to be analyzed;
b) encapsulating each of the said one or more cell types to be analyzed in the same in vitro drug testing assay with a biopolymer of alginate, wherein in the case there are more than one cell type, all, or all but one, of the respective capsules are labelled, each with a different type of fluorophore;
c) co-culturing all the encapsulated cell types to be analyzed of step b) in the same in vitro drug testing assay, optionally with one or more additional unlabeled cell types that are optionally encapsulated with labeled or unlabeled biopolymers of alginate;
d) optionally exposing said co-culture to one or more drugs;
e) measuring the activity of said multiple biological processes in each cell type of step c) to be analyzed in the same in vitro drug testing assay, by individually analyzing through optical imaging, before and/or after exposure to said optional one or more drugs to be tested, the fluorescence or bioluminescent intensities of each fluorescent or bioluminescent reporter gene;

wherein when more than one cell types are co-cultured, each cell type to be analyzed is identified through optical imaging on the basis of the type of fluorophore labelling associated with the alginate capsule containing said cell type to be analyzed.

Another object of the present invention is to provide an in vitro drug testing kit suitable for testing the effect of one or more drugs of interest on multiple biological processes in one or more target cell types, the kit comprising:

at least one ready-to-use microwell plate containing one or more alginate-encapsulated target cell types expressing a fluorescent or bioluminescent reporter gene for each biological process to be analyzed in said one or more target cell types, wherein in case there are more than one cell type, all, or all but one, of the respective capsules are labelled, each with a different type of fluorophore.

Other objects and advantages of the invention will become apparent to those skilled in the art from a review of the ensuing detailed description, which proceeds with reference to the following illustrative drawings, and the attendant claims.

### BRIEF DESCRIPTION OF THE FIGURES

FIG. 1: Pharmacological inhibition of Estrogen Receptor alpha (ERα) by tamoxifen (Tam) vs tamoxifen metabolite (4-hydroxytamoxifen; 4OHT) in breast cancer (BC) cells co-cultured with liver cancer (LC) cells (hepatocytes). BC cells stably expressing luciferase under the control of estrogen receptor alpha (ERα) were cultured with or without LC cells and they were treated with increasing concentrations of Tam or one of its metabolites, 4-OHT for 48 hours. BC cells were lysed and bioluminescence was measured to determine ERα activity, which can be considered as a readout for pro-cancer activity in BC cells. The graph shows the percentage of activity of ERα, with the activity of ERα without inhibitor considered to be 100%, as a function of Log [inhibitor concentration]. Data is representative of 3 independent repeats.

FIG. 2: Quantification of two cell proliferation states (active proliferation and latency) in two different cell types (BC and LC) in co-culture. Proliferation of a co-culture of 3D encapsulated BC cells and LC cells genetically engineered to express the dual fluorescence reporter gene FUCCI. BC cells and LC cells stably expressing the FUCCI reporter (proliferating cells are green, not-proliferating cells are red) were encapsulated in far-red-labeled alginate capsules (BC) or in unlabeled alginate capsules (LC). BC capsules were mixed with LC capsules to prepare the co-cultures and they were incubated with different concentrations of Fetal Bovine Serum (FBS) for 96 hours. The fluorescence intensities of the cells and of the capsules were then measured with a ImageXpress Micro Confocal microscope (Molecular Devices), and processed and analysed with the software MetaXpress (Molecular Devices). The graph shows the fluorescence intensities (arbitrary units) of the reporter genes corresponding either to BC cells or to LC cells. Data is representative of 3 independent replicates.

FIG. 3: Multi-culture of encapsulated LC cells with encapsulated BC cells expressing the dual reporter gene FUCCI, and with encapsulated BC cells expressing the reporter gene zipGFP-Casp3. LC cells were encapsulated in green-labelled alginate capsules, BC cells stably expressing the dual reporter gene FUCCI (active proliferating cells are green, not-proliferating cells (latent) are red) were encapsulated in unlabelled alginate capsules, and BC cells stably expressing the reporter gene zipGFP-Casp3 (all cells are red (reporter expression control) and cells become green upon apoptosis induction) were encapsulated in far-red-labelled alginate capsules. Encapsulated cells were mixed together in a 96-well plate with cover glass bottom and incubated at 37° C. for 96 hours. Image acquisition was performed with an automated confocal microscope and image was directly annotated.

FIG. 4: Quantification of BC cell apoptosis either in monoculture or in multi-culture with LC cells and treated with increasing concentrations of Tamoxifen. LC cells were encapsulated in green-labelled alginate capsules, BC cells stably expressing the dual reporter gene FUCCI (active proliferating cells are green, not-proliferating cells (latent) are red) were encapsulated in unlabelled alginate capsules, and BC cells stably expressing the reporter gene zipGFP-Casp3 (all cells are red (reporter expression control) and cells become green upon apoptosis induction) were encapsulated in far-red-labelled alginate capsules. Encapsulated cells were mixed together, with (multi-culture) or without (monoculture) LC cells, in a 96-well plate with cover glass bottom, treated or not with increasing concentrations of Tamoxifen in the micromolar range, and incubated at 37° C. for 96 hours. Image acquisition was performed with an automated confocal microscope. Cell segmentation and quantification of cell fluorescence intensities were performed with the software MetaXpress (Molecular Devices). For the quantification of apoptosis, cell fluorescence intensities from far-red-labelled capsules were measured and the green fluorescence was normalized with the red fluorescence. The graph shows normalized fluorescence intensities of the zipGFP-Casp3 reporter gene upon treatments with increasing concentrations of Tamoxifen. Data is representative of 3 independent replicates.

FIG. 5: Quantification of BC cell proliferation either in monoculture or in multi-culture with LC cells and treated with increasing concentrations of Tamoxifen. LC cells were encapsulated in green-labelled alginate capsules, BC cells stably expressing the dual reporter gene FUCCI (active proliferating cells are green, not-proliferating cells (latent) are red) were encapsulated in unlabelled alginate capsules, and BC cells stably expressing the reporter gene zipGFP-Casp3 (all cells are red (reporter expression control) and cells become green upon apoptosis induction) were encapsulated in far-red-labelled alginate capsules. Encapsulated cells were mixed together, with (multi-culture) or without (monoculture) LC cells, in a 96-well plate with cover glass bottom, treated or not with increasing concentrations of Tamoxifen in the micromolar range, and incubated at 37° C. for 96 hours. Image acquisition was performed with an automated confocal microscope. Cell segmentation and quantification of cell fluorescence intensities were performed with the software MetaXpress (Molecular Devices). For the quantification of proliferation, cell fluorescence intensities from unlabelled capsules were measured. The graph shows the fluorescence intensities (arbitrary units) of the FUCCI dual-reporter gene upon treatments with increasing concentrations of Tamoxifen. Data is representative of 3 independent replicates.

### DETAILED DESCRIPTION OF THE INVENTION

To Applicant's best knowledge, multiplexed drug testing methods have not been combined with 3D spheroid cultures that simulate solid in vivo tissue structures, nor combined with co-culture models that aim to replicate the multi-organ nature of biological systems such as the human body. Unexpectedly, combining the various cell culture drug testing methods provides unique advantages that are not present in any of the methods that address only one or two of the main limitations of current cell culture drug testing, detailed below.

The advantages of a drug testing methodology that combines multiplexed detection of biological processes, and physical separation of different cell types, includes the obvious simple economic measures of experiment cost, labor, and time. It additionally encompasses non-obvious scientific conceptual advances that are unique to the current art. This includes the ability to identify drugs with potentially disparate effects on cells from distinct human organs. For example, a hepatocyte-metabolized drug may have extremely potent anti-tumor proliferation effects and yet exhibit high toxicity to lung cells at very low concentrations, as well as induce unexpected inflammatory responses from immune cells. Therefore, the ability to simultaneously detect the modulation of multiple biological process in different cell types allows unparalleled high-content screening of drug activity. Thus the present invention offers unique advantages over the prior art because it is possible to down select drug candidates that would have otherwise progressed into the next round of the development process in the absence of simultaneous evaluation of biological processes. An additional favourable feature of the current art is the possibility to identify, more efficaciously, drugs with multiple desirable features such as potent biological activity in the target cells of interest, and low toxicity or inflammation of cells from vital organs.

The encapsulation of cells to prevent direct contact of bacteria or hepatocytes with the majority of other cell types is also advantageous because these cells do not normally come into direct contact with most other cell types in vivo. For example, hepatocytes often metabolize circulating drugs, however these cells do not come into direct contact with neurons or pulmonary cells etc. As previously described, most other cell culture systems do not include a means to separately analyze multiple biological processes in distinct cell types, and the current art is therefore uniquely positioned to detect the effects of chemical entities, tested in a drug screen, on cells from a variety of tissues that together aim to better recapitulate an entire biological system.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the singular forms “a”, “an” and “the” include plural references unless the context clearly dictates otherwise.

The term “comprise” is generally used in the sense of include, that is to say permitting the presence of one or more features or components.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “Multiplexed”, refers to drug testing that is capable of simultaneously detecting multiple biological processes based via their associated chemical or biological tags.

The term “spheroid” refers to a 3D agglomeration of cells in the approximate shape of the sphere.

The term “hepatocyte” refers to any primary hepatocytes or any cell lines derived from healthy or transformed hepatocytes and includes for example liver cancer (LC) cell lines.

“Capsule”, refers to a polymeric sheet or layer produced by the microfluidics device, typically comprising of alginate in the current invention that is used to encapsulate or encase cells. It is known in the art that a capsule is a protective barrier which encloses a cell unit. This term is commonly used in the art to refer to semi-permeable or impermeable structures; in the context of the present invention, microcapsules are semi-permeable and allow the passage of the components of growth media and other reagents, but retain labels and tags in order to allow identification of the cell units.

Microencapsulation, or encapsulation, is the enclosing of a cell unit in a microcapsule.

As used herein, the term “culture conditions” refers to the environment which cells are placed in or are exposed to in order to promote growth or differentiation of said cells. Thus, the term refers to the medium, temperature, atmospheric conditions, substrate, stirring conditions and the like which may affect the growth and/or differentiation of cells. More particularly, the term refers to specific agents which may be incorporated into culture media and which may influence the growth and/or differentiation of cells. This includes supernatants or lysates after the culture of primary cells, cell lines, or microbes, or biological fluids or extracts derived from living organisms.

A “cell”, as referred to herein, is defined as the smallest structural unit of an organism that is capable of independent functioning, or a single-celled organism, consisting of one or more nuclei, cytoplasm, and various organelles, all surrounded by a semipermeable cell membrane or cell wall. The cell may be prokaryotic, eukaryotic, animal or plant, or archaebacterial. For example, the cell may be a eukaryotic cell. Cells may be natural or modified, such as by genetic manipulation or passaging in culture, to achieve desired properties. A stem cell is defined in more detail below, and is a totipotent, pluripotent or multipotent cell capable of giving rise to more than one differentiated cell type. Stem cells may be differentiated in vitro to give rise to differentiated cells, which may themselves be multipotent, or may be terminally differentiated. Cells differentiated in vitro are cells which have been created artificially by exposing stem cells to one or more agents which promote cell differentiation.

The term “cells” is used in its general and broad context and defines cells as being either a cell line, primary cells from an organism or human, or bacterial, fungal, or plant cells. The properties of encapsulated cell culture, such as specific pressure forces exerted on encapsulated cells or the formation of 3D tissue like structures, may favour cell differentiation or growth that is otherwise challenging to attain in vitro. For example, encapsulation allows the enhanced expansion of pluripotent neuronal stem cells or chondrocytes, and facilitates long term maintenance of hepatocytes, thus providing additional translational relevance to the proposed drug testing methodology.

A “group” of cell units (or cells) is a plurality of such units which are not linked together. For example, a cell unit is not a group of cells, but one or more cells clustered together in one single unit. Single cells, and individual cell units, may be pooled to form a group of cells or cell units. Groups can be split, by dividing the groups into two or more groups of cells or cell units.

The terms “drug testing” is a generic term that includes drug screening. According to the invention, drug testing can comprise of screening molecule libraries to identify new chemical entities with the desired biological effect, but can also include testing of known or previously approved drugs in vitro to identify which drugs or drug combinations are the most appropriate for a patient, or to characterize the mechanisms or off-target effects of new or previously identified drugs.

One of the objects of the invention concerns a method for detecting different biological processes e.g. cell proliferation, cell toxicity, neoplastic growth or inhibition, immune cell activation, inflammatory responses, antibiotic resistance, drug synergy, in one or more types of cells grown in 3D cell co-cultures, with or without the addition of test compounds for drug testing. The method uses encapsulated cell lines that have been genetically engineered to contain fluorescent reporter genes that correspond to the different aforementioned biological processes, and the readout of the magnitude of these biological processes can be analysed individually because of the use of different fluorescent colors for each reporter gene. The biological activity can also be distinguished between different cell types by encapsulating different cell types in alginate that is coupled to different fluorescent compounds. The method aims to improve drug screening assays, by (1) incorporating different cell lines, for example hepatocytes that can metabolize drugs together with breast cancer cells used as the cellular drug target, (2) providing simultaneous information about multiple biological readouts in a single test—notably toxicity on liver or other cells, and or anti-neoplastic activity or other metrics of drug efficacy, and (3) using 3D cell culture of target cells, which provides additional physiological relevance when testing drugs for efficacy.

A “label” or “tag”, as used herein, is a means to identify a cell unit and/or determine a culture condition, or a sequence of culture conditions, to which the cell unit has been exposed. Thus, a label may be a group of labels, each added at a specific culturing step; or a label added at the beginning or the experiment which is modified according to, or tracked during, the culturing steps to which the cell unit is exposed; or simply a positional reference, which allows the culturing steps used to be deduced. A label or tag may also be a device that reports or records the location or the identity of a cell unit at any one time, or assigns a unique identifier to the cell unit. Examples of labels or tags are molecules of unique sequence, structure or mass; or fluorescent molecules or objects such as beads; or radiofrequency and other transponders; or objects with unique markings or shapes. Because different fluorochromes may have overlap in their light emission spectra, the selection of different tags must be performed so that there is minimal or no fluorescence overlap between the different fluorochromes, or that the measurements are compensated using staining control samples, so that the interpretation of the imaging results is not compromised.

An “identifying label” is a label which permits the nature of the cell unit to which it is attached to be determined. This allows the exposure of cell units to different culture conditions to be recorded, by addition of an identifying label at each exposure, and subsequently deconvoluted by analysis of the labels.

A cell is “exposed to culture conditions” when it is placed in contact with a medium, or grown under conditions which affect one or more cellular process(es) such as the growth, differentiation, or metabolic state of the cell.

Thus, if the culture conditions comprise culturing the cell in a medium, the cell is placed in the medium for a sufficient period of time for it to have an effect. Likewise, if the conditions are temperature conditions, the cells are cultured at the desired temperature. The “pooling” of one or more groups of cell units involves the admixture of the groups to create a single group or pool which comprises cell units of more than one background, that is, that have been exposed to more than one different sets of culture conditions. A pool may be subdivided further into groups, either randomly or non-randomly; such groups are not themselves “pools” for the present purposes, but may themselves be pooled by combination, for example after exposure to different sets of culture conditions.

“Cell growth” and “cell proliferation” are used interchangeably herein to denote multiplication of cell numbers without differentiation into different cell types or lineages. In other words, the terms denote increase of viable cell numbers. Preferably proliferation is not accompanied by appreciable changes in phenotype or genotype.

“Cell differentiation” is the development, from a cell type, of a different cell type. For example, a bipotent, pluripotent or totipotent cell may differentiate into a neural cell. Differentiation may be accompanied by proliferation, or may be independent thereof. The term ‘differentiation’ generally refers to the acquisition of a phenotype of a mature cell type from a less developmentally defined cell type, e. g. a neuron, or a lymphocyte, but does not preclude trans-differentiation, whereby one mature cell type may convert to another mature cell type e. g. a neuron to a lymphocyte, or a monocyte to a macrophage.

The term “plurality” means more than one. In the context of labels, it describes the fact that each encapsulation allows at least one more label to be added, such that a multiply-encapsulated cell unit can be labelled with at least one label per encapsulation. In the context of cell units within microcapsules, two, three, four, five, six or more cell units can be included in a single micro-capsule.

The terms “phenotypic screening” refers to a type of screening used in biological research and drug discovery to identify substances such as small molecules, peptides, antibody or RNAi that alter the phenotype of a cell or an organism in a desired manner.

Fluorescent reagents are commonly used in Life Sciences laboratories to measure the activities of biological processes. For instance, combination of the fluorescent reagents DiOC6 and propidium iodide allows the detection and quantification of living (green fluorescence) and dead cells (red fluorescence) in a cell population. Dihydroethidium is a fluorescent probe to measure the production of reactive oxygen species in cells, which is an indicator of cellular stress, by becoming red into the cells after being oxidized. Coumarin boronate or fluorescein-boronate can be used to measure the production of nitric oxides into the cells, which are involved in many physiological processes including inflammation, by emitting blue or green fluorescence, respectively. Fura-2 is a fluorescent probe to measure the intracellular concentration of calcium which fluctuates upon stimulation of signal transduction pathways.

Incorporation of such reagents in the present invention helps to expand the spectra of biological activities that can be analyzed. However, instead of reporter genes, fluorescent reagents indifferently report a biological activity in all cells of a cell population. Indeed, fluorescent reagents are soluble molecules that freely diffuse after addition in the cell culture medium, thus staining all cells. Fluorescent reagents can be used in combination with fluorescent or bioluminescent reporter genes to measure an additional biological activity, sometimes for which no reporter gene may exist. To do so, fluorescent reporter gene activities have to be measured first because they specifically report biological activities in the cell types where they are expressed. Addition of the fluorescent reagent can then be performed to stain all cells. Colors of capsules can still be used to separately measure the fluorescence of the reagent, and thus to measure the related biological activity, in a specific encapsulated cell type in the cell co-culture. Also, several fluorescent reagents can be simultaneously used until their fluorescence spectra do not overlap.

In particular, one of the objects of the present invention is to provide an in vitro method for drug testing that is capable of analyzing multiple biological processes in one or more cell types, the method comprising:
a) expressing in each of said one or more cell types to be analyzed in the same in vitro drug testing assay, at least one fluorescent or bioluminescent reporter gene for each biological process to be analyzed;
b) encapsulating each of the said one or more cell types to be analyzed in the same in vitro drug testing assay with a biopolymer of alginate, wherein in the case there are more than one cell type, all, or all but one, of the respective capsules are labelled, each with a different type of fluorophore;
c) co-culturing all the encapsulated cell types to be analyzed of step b) in the same in vitro drug testing assay, optionally with one or more additional unlabeled cell types that are optionally encapsulated with labeled or unlabeled biopolymers of alginate;
d) optionally exposing said co-culture to one or more drugs;
e) measuring the activity of said multiple biological processes in each cell type of step c) to be analyzed in the same in vitro drug testing assay, by individually analyzing through optical imaging, before and/or after exposure to said optional one or more drugs to be tested, the fluorescence or bioluminescent intensities of each fluorescent or bioluminescent reporter gene;

wherein when more than one cell types are co-cultured, each cell type to be analyzed is identified through optical imaging on the basis of the type of fluorophore labelling associated with the alginate capsule containing said cell type to be analyzed.

In addition to testing drugs, the in vitro method of the invention is also adapted to test biological fluids including supernatants from cells or tissues, or lysates from cells or microbial broths.

Advantageously, the present invention is also intended to drug testing but other families of molecules with anticipated or confirmed effects on human health can be tested. For example, the present invention can be used to predict the effects of unknown compounds on human health and/or to expand knowledge of known compounds by choosing the relevant biological activities and cell types to be tested. Nutrients, cosmetics, pollutants and other chemicals such as exogenous active substances can be tested until they can be added in a soluble form in a cell culture medium. The present invention can also be used for testing fluids with undetermined and/or complex composition like polluted liquids (e.g. water downstream chemical factories or hospitals etc. . . . ) or physiological fluids (e.g urine, serum etc. . . . ) to determine their effects on specific biological activities and cell types, without needing to identify the one or several molecules they contain and responsible for the measured effect. With such applications, the present invention can be globally used as a predictive tool to anticipate the potential harmful and/or beneficial effects of compounds and/or fluids on human health.

Biological activities also defined as multiple biological processes can be reported both by fluorescent reporter genes or bioluminescent reporter genes. Several bioluminescent proteins, called luciferases, can be used as reporter genes. For example, firefly luciferase, Renilla luciferase, and Gaussia luciferase are commonly used as bioluminescent reporter genes. Bioluminescence is measured with bioluminometers and it is often more sensitive than fluorescence. Several reporter genes are only available with a bioluminescent reporter gene because they report weak biological activities that are otherwise poorly detected with fluorescent reporter genes. Bioluminescent reporter genes thus represent a good alternative to fluorescent reporter genes for the measurement of poorly detectable biological activities. Because of the limited set of available bioluminescent colors, multiplexing with several bioluminescent reporter genes is difficult and fluorescent reporter genes are often preferred.

However, dual-reporter systems can be designed with the firefly luciferase and the renilla where the bioluminescence of both bioluminescent reporter proteins can be sequentially measured on the same sample. A possibility is to combine fluorescent reporter genes with bioluminescent reporter genes but fluorescence must be measured first before measuring bioluminescence. Indeed, the bioluminescent signal is very strong and impairs the detection of fluorescence. Bioluminescence can be easily controlled because a substrate is needed and can thus be added in the cell co-culture just after fluorescence acquisition. Another possibility is to use a single bioluminescent reporter gene in a cell type in co-culture with other cell types to measure a specific biological activity while considering possible biological communications between those cell types that could influence the specific biological activity reported (see Example 1).

The invention encompasses the possibility of encapsulating several cell types per capsule. For example it is possible to prepare a liver capsule with hepatocytes alongside other cells normally found in the liver, to better recapitulate liver functions in vitro, and thus metabolize drugs or investigate hepatotoxicity in a way that is more reflective of in vivo biology. Alternatively it is possible to generate spheroids containing both cancer cells (tumor cell line or patient derived tumor cells) together with immune system or stromal cells (e.g. cell lines of monocytes, T lymphocytes, or endothelial cells, or alternatively patient derived primary cells) within the a single capsule to test for drugs that can (re)program non-cancerous tumor-associated cells to favour tumor regression, e.g. for tumor-associated immune cells to become more inflammatory or initiate cytotoxic immune responses against the tumor, or for tumor endothelial cells to reduce their secretion of angiogenic factors.

Thus, the possibility of using several types of cells per capsule is particularly interesting when the different cells are recognizable (i.e. one cell type in green, another cell type in red, or one biological activity readout in green and a second biological activity in red, both in the same capsule).

Preferably the fluorophore is coupled to an amine-alginate biopolymer, however, a wide variety of fluorescent dyes with simple covalent chemistries are known, and these could substitute the amine-based dyes if they have more desirable physicochemical or assay-dependent properties, or better cross compatibility with other fluorescent labels when combining higher numbers of fluorophores in the same test (e.g. 4 different fluorescent capsule labels, plus one unlabeled capsule, and 4 different reporter gene fluorescent labels), that can all be accurately distinguished from one another via a given filter and laser configuration of an imaging microscope.

According to the invention, the multiple biological processes are selected from the list comprising: cell proliferation, inflammation, tumor growth or inhibition, drug toxicity, angiogenesis, immune activation, detoxification responses, hormone response, xenobiotic response, genotoxic stress response and apoptosis.

In accordance with one embodiment of the invention, said one or more cell types are selected for co-culturing based on anticipated or confirmed physiological interactions between said cell types, and/or based on anticipated or confirmed effects of one or more of said cell types on the one or more drugs to be tested.

For example, the anticipated or previously described physiological interactions between one or more cell types may comprise the co-culture of hepatocytes with a cancer cell type based on the anticipation that a drug can be metabolized by hepatocytes, changing the reactivity of the drug towards the targeted cancer cell type, or the co-culture of ovarian cancer cells with breast cancer cells based on the knowledge that ovarian cancer cells produce estrogen that induce the proliferation of breast cancer cells, or alternatively the co-culture of hepatocytes with endometrium cancer cells and with breast cancer cells to test antiestrogen drug based on the knowledge that a drug being antiestrogen in breast cancer cells can be pro-estrogenic in the endometrium which is can be an anticipated adverse side effect of antiestrogen drugs.

According to a preferred embodiment of the invention, the in vitro method for drug testing comprises step d) in which said co-culture is exposed to one or more drugs.

In particular, the in vitro method for drug testing that is capable of analyzing multiple biological processes in one or more cell types, comprises the steps of:
a) expressing in each of said one or more cell types to be analyzed in the same in vitro drug testing assay, at least one fluorescent or bioluminescent reporter gene for each biological process to be analyzed;
b) encapsulating each of the said one or more cell types to be analyzed in the same in vitro drug testing assay with a biopolymer of alginate, wherein in the case there are more than one cell type, all, or all but one, of the respective capsules are labelled, each with a different type of fluorophore;
c) co-culturing all the encapsulated cell types to be analyzed of step b) in the same in vitro drug testing assay, optionally with one or more additional unlabeled cell types that are optionally encapsulated with labeled or unlabeled biopolymers of alginate;
d) exposing said co-culture to one or more drugs;
e) measuring the activity of said multiple biological processes in each cell type of step c) to be analyzed in the same in vitro drug testing assay, by individually analyzing through optical imaging, before and/or after exposure to said one or more drugs to be tested, the fluorescence or bioluminescent intensities of each fluorescent or bioluminescent reporter gene;

wherein when more than one cell types are co-cultured, each cell type to be analyzed is identified through optical imaging on the basis of the type of fluorophore labelling associated with the alginate capsule containing said cell type to be analyzed.

According to a preferred embodiment, the in vitro method for drug testing further comprises an additional step f) in which said co-culture is exposed to one or more fluorescent reagents to measure one or more additional biological processes.

Preferably, optical imaging is performed by microscopy and/or luminometer. Optical imaging may be performed by flow cytometry given an appropriate instrument nozzle is used and cell capsule sizes are sufficiently small. The use of flow cytometry may allow increased multiplexing of the assay because more fluorescence parameters can readily be analyzed compared to standard imaging microscopes, and could be analyzed without the use of image analysis software, however the low number of capsules per plate well is more suited to image-based microscopy.

According to another embodiment of the invention, said at least one or more cell types are selected from the group comprising cell lines or primary tissue cell subsets of: hepatocytes, tumor cells, brain cells, epithelial cells, endothelial cells, immune system cells, pluripotent stem cells, embryonic stem cells, lung cells, kidney cells, artery cells, bone cells, cartilage cells, muscle cells, pancreatic cells, intestinal cells, skin cells, fibroblasts, fungal cells or bacteria.

Preferably, at least one of said one or more cell types to be analyzed in the in vitro drug testing assay or at least one of the optionally added unlabeled cell types of step c) are hepatocytes.

According to an embodiment of the invention, the at least one of said one or more type of encapsulated cell derives from a patient cancer sample.

According to an embodiment of the invention, the at least one of said one or more cell types is a bacterium or a fungal cell. In the latter, the drug to be tested is an antibiotic or a fungicide. According to this embodiment, the multiple biological processes to be analyzed is selected from the list comprising: anti-microbial resistance or susceptibility, bacterial proliferation, bactericidal or bacteriostatic activity.

In another embodiment, the in vitro method of the invention is designed for drug screening or testing, wherein said multiple fluorescent reporter genes are selected for biological processes that are investigated during phenotypic drug discovery process.

For example, the gene reporter is selected because it is a specific target that has previously been identified via mechanistic biological studies, i.e. a receptor with important roles in the pathogenesis of particular cancers or other specific diseases.

Preferably, the gene reporter and regulatory element are flanked by CTCF insulator sequences.

The choice of fluorochromes is such that spectral overlap between and amongst the alginate fluorophores and fluorescent reporter gene constructions is minimized or negated using proper controls when configuring the detection equipment (microscope or cytometer).

In other words, an object of the invention consists of fluorescent reporter gene-expressing eukaryotic (e.g. mammalian cells) or prokaryotic cells (e.g. bacteria) encapsulated in fluorescently-labelled alginate capsules. The combination of these two different types of fluorescent labelling (intracellular reporter gene and alginate capsule labelling) allows the simultaneous monitoring of multiple biological processes in one or more cell types.

Specifically, cells are encapsulated with a biopolymer of alginate. The capsule exerts a physical constraint on the cells like the normal physiological pressure. This pressure forces the multicellular aggregate inside the capsule to stay at a size below 300 μm compatible with the diffusion of oxygen and nutrients through the cells, increasing the physiological relevance of this cell culture model (examples of increased biological relevance is listed above, neuronal stem cells and chondrocytes).

Each cell type is encapsulated in alginate shells chemically modified with one specific fluorophore. Therefore, each fluorophore allows for the identification of capsules containing one specific cell type during the analysis of an in vitro drug test.

The encapsulated cells will typically consist of cell lines that are genetically engineered to express different fluorescent reporter molecules, or other reporter molecules, which allow the monitoring of key activities involved in tested “biological processes”.

The panel of genes that can be simultaneously monitored includes key regulators or biomarkers of commonly investigated features i.e. biological processes, which can be detected in a drug test. These features include cell toxicity, cell proliferation, inflammation, hormone response, xenobiotic response, genotoxic stress response, and antibiotic resistance.

In particular, the invention comprises of a method to characterize the effects of drugs on multiple cellular processes in multiple cell types, simultaneously within the same test. To achieve this, the method employs encapsulated cells which have been genetically engineered so that one or more cellular process can be monitored via a regulatory element that is fused to a fluorescent reporter gene whose fluorescent activity are indicative of a biological process activity. Cell lines used in the co-culture can be engineered to express one or more reporter genes that are involved in biological processes, including but not limited to: cell proliferation, xenobiotic stress response, inflammation, tumor growth or inhibition, and drug toxicity or detoxification responses.

The same cell type can be genetically engineered to express reporter genes for different biological processes. To multiplex the analysis of multiple reporter genes, cells are divided into the required number of separate batches of cells, and each batch is engineered to contain a single reporter gene corresponding to a single biological process. The separate batches of cells are either pooled together to be encapsulated in the same capsule, or they are each encapsulated separately, to measure the activity of multiple biological processes either in mixed co-culture, or in physically separated co-culture, respectively). Multiplexed analysis of genes that correspond to different cellular processes is intended to provide many benefits including increased ability to predict the best drug candidates and economy of reagents, consumables, and time required to perform in-depth characterization of drug candidates.

Each type of capsule contains one or more cell types that are normally found in a single human organ or distinct physiological site such as a tumor. Encapsulation of cells involves the preparation of separate single cell suspensions of different cell types. The suspensions are then encapsulated in a thin layer of alginate which has been previously labelled with one of many fluorescent markers that allows the identification of capsule contents by microscopy.

Encapsulated cells are then incubated to facilitate the formation of 3D tissue-like structures called spheroids. Different encapsulated cell spheroids can then be mixed to create a co-culture of different cell subtypes, which may include but are not limited to, tumor cell lines, patient derived primary tumor cells, and cell lines—particularly hepatocytes which can metabolize drugs.

The use of encapsulated 3D cell cultures (i) prevents cell to cell contact of different cell types which are not typically found in the same human organ, and (2) enables the simultaneous analysis of different cell types within the same well, for example the toxicity of a drug can be separately evaluated on hepatocytes and cancer cells within the same co-culture.

The aim of co-culturing multiple cell types within the same well is to recapitulate multicellular microenvironments, tissues, or inter-organ systems that occur in the human body, in order to increase the clinical relevance of this drug testing approach. For example, co-culture of tumor cells and hepatocytes in separate capsules aims to simulate common processes after the administration of drugs in vivo, where the liver first metabolizes the drug and then drug metabolites are the main effectors of anti-tumor responses in distal anatomical sites.

The invention is also time and resource saving because multiple effects can be investigated simultaneously, for example anti-tumor effects as well as liver toxicity. This is important because drug-induced liver injury (DILI) is one of the main reasons that many drug candidates do not progress through the development process.

One unique advantage of the present invention relies in the ability to measure multiple biological processes in a co-culture of mixed cell types, which increases the efficiency and accuracy (more translational relevance to in vivo models) of drug testing.

Shared advantages of the current invention with other 3D co-culture or encapsulated cell-based drug testing methodologies is to increase the clinical relevance of the drug test via the following features, which are common to existing technologies:
Alginate capsules act as a physical separation of different cell types that do not normally contact each other in vivo
3D spheroid cell cultures behave more like in vivo tissue compared to cell culture monolayers

Further, the invention allows an increase in the number of biological processes that can be investigated simultaneously, as well as an increase in the repertoire of biological processes i.e.

the creation of a library of reporter genes to quantitate a large portion of biological process that are commonly investigated by pharmaceutical companies.

The present invention combines many existing individual elements or processes that are currently known in the field of cell biology and applied drug discovery, as well as elements that are completely novel. These technical elements or processes are described below:
a. Encapsulation of cells to form 3D cell spheroids with tissue-like properties. Preferably, the method of alginate-based encapsulation of cells is performed with a microfluidics device. The microfluidics device is described in Alessandri K, Feyeux M, Gurchenkov B, Delgado C, Trushko A, Krause K H, Vignjević D, Nassoy P, Roux A. Lab Chip. 2016 Apr. 26; 16(9):1593-1604. doi: 10.1039/c61c00133e.
b. Co-culture of different cell types to produce in vivo-like cellular interactions, which helps to achieve more clinically relevant results in drug screening. Preferably the invention uses hepatocytes to metabolize drugs in cell co-cultures.
c. Labelling of cell capsules with fluorescent probes. Techniques used to label alginate with common fluorescent molecules are widely known in the art.
d. Genetic engineering and detection of multiple regulatory activities that are related to major cellular processes. The invention concerns multiple simultaneous (multiplexed) analysis of the fluorescent intensities of different fluorescent reporter genes within the same cell. The method of the invention aims to perform ‘phenotypic drug screening’, which does not rely on knowing the identity of specific molecular drug targets, or alternatively ‘target-based’ drug screening, where the regulatory activity of a specific protein is measured in cells. This element represents a unique feature of the present invention.

Another object of the present invention is to provide an in vitro drug testing kit suitable for testing the effect of one or more drugs of interest on multiple biological processes in one or more target cell types, the kit comprising:
at least one ready-to-use microwell plate containing one or more alginate-encapsulated target cell types expressing a fluorescent or bioluminescent reporter gene for each biological process to be analyzed in said one or more target cell types, wherein in case there are more than one cell type, all, or all but one, of the respective capsules are labelled, each with a different type of fluorophore.

Preferably, at least one of said one or more target cell types are hepatocytes.

According to an embodiment of the invention, said fluorophore-labelled alginate-encapsulated one or more target cell types are co-cultured with one or more patient-derived cell types. Preferably, the one or more patient-derived cell types are genetically engineered to contain at least one fluorescent reporter gene for each multiple biological process to be analyzed.

More preferably, the one or more patient-derived cell types are each encapsulated with a biopolymer of alginate labelled with a fluorophore.

One advantage of the in vitro drug testing kit is that it can be used for phenotypic (affects general biological processes with common reporters for nearly all cells) or target-based (modulation of specific pathways that might only be present in some cells e.g. estrogen receptor, and have been previously identified as ideal therapeutic targets) drug discovery.

According to another embodiment of the invention, the multiple biological processes to be analyzed are selected from the list comprising: cell proliferation, inflammation, tumor growth or inhibition, drug toxicity, angiogenesis, immune activation, detoxification responses, hormone response, xenobiotic response, genotoxic stress response and apoptosis.

According to a further embodiment of the invention, the one or more patient-derived cell types are selected from the list comprising: hepatocytes, tumor cells, brain cells, epithelial cells, endothelial cells, immune system cells, pluripotent stem cells, embryonic stem cells, lung cells, kidney cells, artery cells, bone cells, cartilage cells, muscle cells, pancreatic cells, intestinal cells, fibroblasts or skin cells.

In accordance with one embodiment of the invention, one or more drugs are tested per well of said at least one ready-to-use microwell plate to identify the effects of said one or more drugs on biological processes within said encapsulated target cell types.

In accordance with one another embodiment of the invention, more than one drug are tested per well of said at least one ready-to-use microwell plate to identify interactions between said drugs.

In particular, one or more drugs are tested per well to identify the effects of a drug on biological processes within encapsulated cells of interest, or alternatively interactions between multiple drugs e.g. synergy or dysergy of the modulation of biological processes including but not limited to cell cytotoxicity or proliferation.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

Description:

A monolayer of Breast Cancer (BC) cells stably expressing a luciferase reporter gene under the control of an Estrogen Response Element (MELN cells, Table 1 and 2) is co-cultured or not with encapsulated Liver Cancer (LC) cells and treated with increasing concentrations of Tamoxifen or 4-hydroxytamoxifen for 48 hours. The activity of Estrogen Receptor a in BC cells is determined by measuring the activity of Luciferase (bioluminescence intensity).

Material and Methods:

Cell Seeding for BC Cells

Aspirate the culture medium on the MELN cells (see Table 1)

Wash once with PBS and then aspirate the PBS

Add trypsin on the cells and aspirate the excess of trypsin

Incubate 5 minutes at 37° C.

Repeatedly pipette white cell culture medium (DMEM without phenol red 10% charcoal-stripped foetal bovine serum) over the cells to detach and then collect them into a tube Seed the cells in a 96-well plate with 10000 cells/well in white cell culture medium. Incubate the cells at 37° C.

TABLE 1
Characteristics of the cell lines used in encapsulated 3D
cell co-culture for the proof of concept experiments

| Cell line name | Organ | Tissue Origin | Supplier | Cell Culture Medium |
|---|---|---|---|---|
| MELN | Breast | Ductal carcinoma | Gift | DMEM 10% Fetal |
| | | | (Patrick Balaguer) | Bovine Serum |
| MDA-MB-134 | Breast | Ductal carcinoma | ATCC | DMEM 10% Fetal |
| | | | | Bovine Serum |
| HepG2 | Liver | Hepatocellular | ATCC | DMEM 10% Fetal |
| | | carcinoma | | Bovine Serum |

Protocol for the Encapsulation of LC Cells:

Preparing Cells for Encapsulation:

Wash LC cells (see Table 1) from a confluent dish with PBS, detach via trypsin treatment and collect them using cell culture medium

Count the cells and prepare a cell suspension with 5 million cells per ml.

Pour the cell suspension through a 40 μm Corning filter into a 50 ml Falcon tube.

Put 1 ml of cell suspension (i.e. 5 million of cells) into a 1.5 mL centrifuge tube

Centrifuge cells at 1000 rpm for 5 minutes to pellet them and resuspend them in Sorbitol 300 μM (90 μl). Keep the cells on ice.

Encapsulation:

Encapsulation is performed with a patented device (WO/2013/113855) as described in Alessandri K, Feyeux M, Gurchenkov B, Delgado C, Trushko A, Krause K H, Vignjević D, Nassoy P, Roux A. Lab Chip. 2016 Apr. 26; 16(9):1593-1604. doi: 10.1039/c61c00133e. Collect capsules into a 10 cm petri dish containing calcium solution, Incubate 30 minutes Replace the calcium solution with cell culture medium

Waiting for the capsules to settle at the bottom of the dish

Aspirate 90% of the calcium solution using an 18-gauge needle, avoiding aspirating the capsules at the bottom of the dish.

Fill 10 cm capsule dish fully (40 mL) with cell culture medium.

Allow capsules to settle, aspirate and refill with culture media. Repeat twice, leaving 15 ml of cell culture medium on the last repeat.

Incubate Capsules are incubated at 37° C.

Loading of LC Capsules on Plate and Treatments

Aspirate the medium of the capsules, leaving approximately 5 ml of medium.

With a 1 ml pipet equipped with tips that allow the capsules to pass through, wash the dish surface with the remaining medium

Collect as many capsules as possible in 15 ml tubes.

Add 5 ml of medium to collect the remaining capsules with a 1 ml pipet.
Centrifuge the capsules at 300 rpm for 1 minute, aspirate the medium and resuspend the capsules with 10 ml of cell culture medium without phenol red (DMEM without phenol red 10% charcoal-stripped foetal bovine serum). Repeat this once more.
Centrifuge the capsules at 300 rpm for 1 minute, aspirate the medium and leave approximately 200 μl of medium at the top of the capsules.

Dispense capsules at the top of the BC cell monolayer in the 96-well plate by continuously pipetting up and down to maintain the capsules in suspension before transferring the proper volume into the well.

Half of wells are not filled with capsules (conditions with BC cells only) Treat all wells with 10 pM of 17β-Estradiol (E2), and then add different concentrations of either Tamoxifen or 4-hydroxytamoxifen ranging from 30 pM to 1 μM.

Incubate the plate at 37° C. for 48 hours.

Measurement of Luciferase Activity

Aspirate the capsules, wash once with PBS and aspirate to remove completely the capsules Add 20 μl of Passive Lysis Buffer (Promega) on BC cells and incubate on a plate shaker for 15 minutes at room temperature at 100 rpm

Transfer 10 μl of the lysates in a white 96-well plate adapted for luminometer and add 10 μl of Luciferase substrate (Promega)

Read the bioluminescence intensity with a luminometer.

Results:

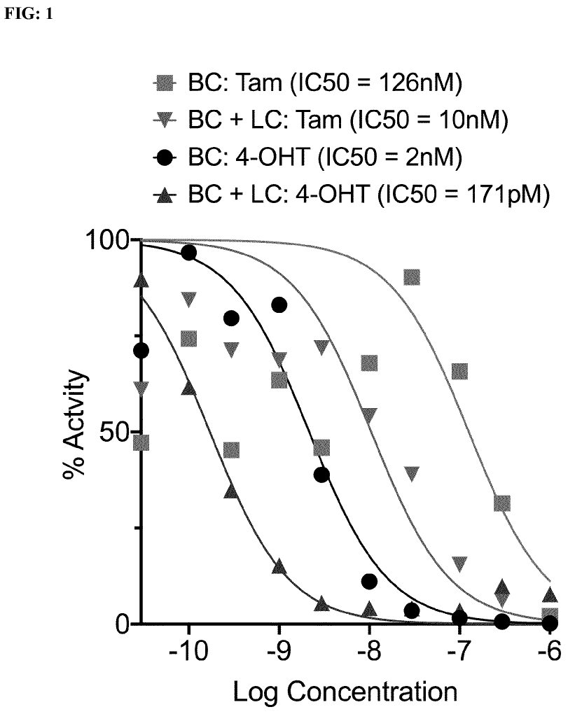

In the FIG. 1, we observe that the inclusion of encapsulated LC cells in an in vitro co-culture with BC cells growing in monolayer increased the anti-cancer activity of Tamoxifen (inhibition of ERα activity, IC50 is lower when LC cells are included). This suggests that the co-culture of BC cells with encapsulated LC cells can recapitulate the physiological production of secondary active drug metabolite (endoxifen) by LC cells, which may otherwise have been overlooked in a mono-culture system including only the pro-drug (Tamoxifen) and BC target cell line—see FIG. 1 for details.

### Example 2

Description:

A co-culture of encapsulated BC cells stably expressing the dual-reporter FUCCI (see Table 2) in far-red-labeled capsules and encapsulated LC cells stably expressing the dual-reporter FUCCI in unlabeled capsules incubated with different serum concentrations. Amounts of active proliferating cells and not-proliferating cells (latent) are determined by quantifying the proportion of red (G1 phase) and green (G2/S phase) nuclei both in LC cells and in BC cells simultaneously in the same co-culture wells.

Material and Methods:

Protocol for Establishing a Stable Reporter Cell Line:

Seed HEK293 cells in 10 cm-dishes at 4 million cells/dish.

Incubate for 24 hours at 37° C.

Cells are transfected with a mix of the plasmids pMD2G, psPAX2 and the lentiviral reporter plasmid of interest (pBOB-EF1-fastFUCCI-puro) by using a PolyEthylenelmine transfection method.

Add the transfection solution to the cells, and incubate overnight at 37° C.

Perform a complete medium change.

Incubate cells at 37° C. for 8 hours.

Collect the medium, which contains lentiviruses, into a tube.

Add complete medium to the cells and incubate the cells at 37° C. for 16 hours.

Repeat the collection with media twice more at 8 to 16-hour intervals.

Cells used to generate the stable reporter cell line (MDA-MB134 and HepG2, Table 1) are seeded directly into the lentiviruses-containing medium, with the same density as for normal cell culture, at 37° C. for 96 hours.

Aspirate Lentiviruses-containing medium and replace with complete cell culture medium.

Add puromycin at a final concentration of 2 μg/mL directly into the culture medium for 24 hours, which selects for antibiotic resistant (reporter+) cells.

Remove puromycin culture medium containing dead cells and replace with complete cell culture medium with puromycin

The stable reporter cell line is maintained in a medium with 1 μg/mL of puromycin.

TABLE 2
Biological activities and their related reporter gene features.

| Biological | | | |
|---|---|---|---|
| process | Reporter system | Readout | Source |
| Prolifer- | Fluorescent Ubiquitination- | Green (λ Ex/Em: | Addgene: |
| ation | based Cell Cycle Indicator | 492/505) and red | plasmid |
| | (FUCCI). Not-proliferating | (λ Ex/Em: | #86849 |
| | cells are red while | 551/565) | |
| | proliferating cells are green. | fluorescence | |
| | | intensities. | |
| Apoptosis | zip Green Fluorescent | Green (λ Ex/Em: | Addgene: |
| | Protein-Caspase3 (zipGFP- | 485/510) and red | plasmid |
| | Casp3). Cells expressing the | (λ Ex/Em: | #81241 |
| | reporter are red, and cells | 587/610) | |
| | undergoing caspase- | fluorescence | |
| | dependent apoptosis are | intensities. | |
| | green. | | |
| Estrogen | Estrogen Response Element- | Biolumin- | Bunone |
| response | Luciferase. Bioluminescence | escence | et al., |
| | intensity is positively linked | | 1996 |
| | to the activity of estrogen | | |
| | response | | |

Staining of Alginate with Fluorophore Bearing an Amine Group (See Table 3)

Prepare 25 mL of 1% (w/v) Alginate solution in 0.1M IVIES pH 6.0 and mix overnight on a rotator at room temperature (RT).

Add 1 mg of the dye that is dissolved in 200 μl DMSO and let mix with the alginate for 10-30 minutes RT on a rotator.

Add sulfo-NHS dissolved in 200 ul 0.1 M IVIES pH 6.0 for a final concentration of 2 mM in the alginate solution and mix for 30 min RT on a rotator.

Add EDC dissolved in 200 ul 0.1M IVIES pH 6.0 for a final concentration of 5 mM in the alginate solution and let mix and react for 1-2 hours on a rotator at room temperature

Dialyse in a Slide-A-Lyser cassette (10K) in 1 L of distilled water for 30 minutes. Then, change the water, put the cassette in 5 L of distilled water and leave overnight with gentle stirring at 4° C.

Collect the stained alginate from the cassette and transfer it into a plastic tube, store it at 4° C.

Before encapsulation, mix the stained alginate with the unstained alginate (prepared by doing step 1 only) with a stained: not stained ratio of 1:10.

TABLE 3
Characteristics of the fluorochromes used to label alginate capsules.

| | | | λ Ex/Em | | Product |
|---|---|---|---|---|---|
| Fluorochrome | Chemical name | Color | (nm) | Reference | Supplier |
| ATTO 647N Amine | Protected by a license | Far-red | 646/664 | 95349 | Sigma-Aldrich |
| Fluoresceinamine, | 5-aminofluorescein | Green | 496/517 | 201626 | Sigma-Aldrich |
| Isomer I | | | | | |

Protocol for the Encapsulation of Stable Reporter Cells:

Preparing Cells for Encapsulation:

Same protocol as for Example 1 “Preparing cell for encapsulation”.

Encapsulation:

Same protocol as for Example 1 “Encapsulation” except that Applicants use far-red-labelled alginate to encapsulate BC cells and unlabelled alginate to encapsulate LC cells.

Loading of BC and LC Capsules on Plate to Make the Co-Culture

Aspirate the medium of the capsules, leaving approximately 5 ml of medium.

With a 1 ml pipet equipped with tips that allow the capsules to pass through, wash the dish surface with the remaining medium.

Collect as many capsules as possible in 15 ml tubes.

Add 5 ml of medium to collect the remaining capsules with a 1 ml pipet.
Centrifuge the capsules at 300 rpm for 1 minute, aspirate the medium and resuspend the capsules with 10 ml of cell culture medium.
Centrifuge the capsules at 300 rpm for 1 minute, aspirate the medium and leave approximately 200 μl of medium at the top of the capsules.

Dispense capsules in wells of a 96-well imaging quality plate by continuously pipetting up and down to maintain the capsules in suspension before transferring the proper volume into the well.

Foetal bovine serum is added or not to the wells and co-cultures are incubated at 37° C. for 96 hours.

Measurement of the Fluorescence Intensities of BC and LC Cells in Co-Culture

Image acquisition of the 96-well plate containing the encapsulated 3D cell co-culture is performed with an automated confocal microscope (IXM-C, Molecular Devices™). A 4× objective is used and 10 z-steps are performed on 12 different areas per well for each fluorescent channel.

The method used here is then a typical High Content Screening (HCS) procedure, which is now the method of choice in the pharmaceutical industry for drug discovery. After image acquisition, an image analysis is developed in two steps: first, the identification of the different cell reporters and capsules in the image, by applying series of segmentation process which generate relevant object masks; second, several parameters (e.g. fluorescence intensities, size, shape etc. . . . ) are extracted from the original images on which the masks are applied. This image analysis process allows a precise quantification of each proliferation status (reported by the FUCCI reporter) simultaneously in both co-cultured cell types.

Results:

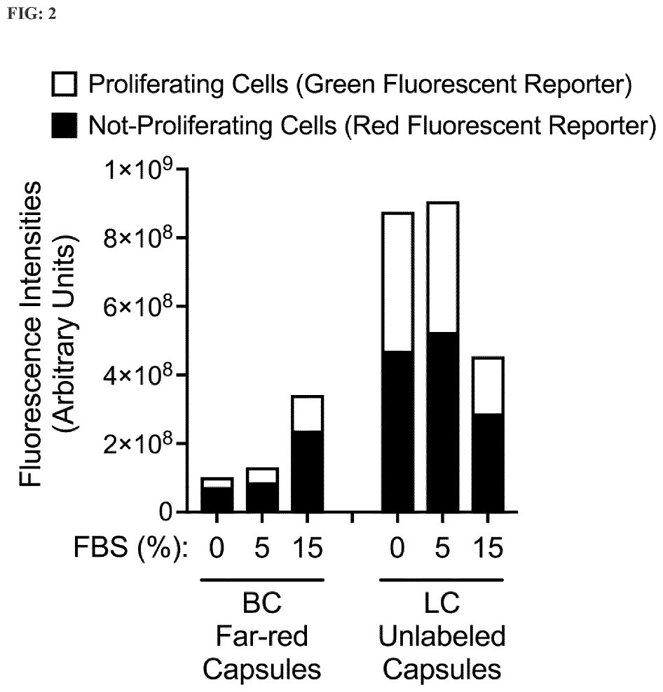

FIG. 2 demonstrates that two different cellular states reported by two different fluorescent reporters (green fluorescent reporter gene for proliferating cells; red fluorescent reporter gene for not-proliferating cells, both included in the FUCCI reporter) can be quantified simultaneously in a 3D co-culture of two different cell types encapsulated in two different alginate capsules (far-red-labelled alginate capsules for BC cells; unlabelled alginate capsules for LC cells). Results from FIG. 2 also show that the fluorescence of reporters can be discriminated between them and with the fluorescence of the labelled alginate capsules by the HCS procedure: automated microscopy acquisition followed by image processing and analysis with the software MetaXpress (Molecular Devices).

### Example 3

Description:

A co-culture of encapsulated BC cells stably expressing the dual-reporter FUCCI (Table 2) in unlabelled capsules, and encapsulated BC cells stably expressing the reporter zipGFP-Casp3 (Table 2) in far-red-labelled capsules, co-cultured or not with encapsulated LC cells in green-labelled capsules and treated with increasing concentrations of Tamoxifen for 96 hours. Induction of apoptosis is measured by automated confocal microscopy (HCS) in BC cells stably expressing the reporter zipGFP-Casp3 and proliferation is measured at the same time in BC cells stably expressing the dual-reporter FUCCI.

Materials and Methods

Protocol for Establishing the Two BC Stable Reporter Cell Lines:

Same protocol as for Example 2 “Protocol for establishing a stable reporter cell line” except that in addition to make a BC cell line stably expressing the dual-reporter FUCCI, Applicants have also made a BC cell line stably expressing the reporter zipGFP-Casp3 (Table2) with a home-made lentiviral construct that contains the reporter integrated into the pHAGE lentiviral vector (pHAGE-fEF1-zipGFP-Casp3).

Staining of Alginate with Fluorophore Bearing an Amine Group (See Table 3)

Same protocol as for Example 2 “Staining of alginate with fluorophore bearing an amine group (see Table 3)”

Protocol for the Encapsulation of Stable Reporter Cells:

Preparing Cells for Encapsulation:

Same protocol as for Example 1 “Preparing cell for encapsulation”

Encapsulation:

Same protocol as for Example 1 “Encapsulation” except that we use unlabelled alginate to encapsulate BC cells stably expressing the dual-reporter FUCCI, far-red-labelled alginate to encapsulate BC cells stably expressing the reporter zipGFP-Casp3, and green-labelled alginate to encapsulate LC cells.

Loading of the Capsules on Plate to Make the Co-Cultures and Treatments

Same protocol as for Example 1 “Loading of LC capsules on plate and treatments” except that:

Dispense capsules in wells of a 96-well imaging quality plate by continuously pipetting up and down to maintain the capsules in suspension before transferring the proper volume into the well.

Half of wells are not filled with LC capsules (conditions with BC cells only)

Treat all wells with 10 pM of 17β-Estradiol (E2), and then add different concentrations of either Tamoxifen or 4-hydroxytamoxifen ranging from 1 μM to 10 μM.

Incubate the plate at 37° C. for 96 hours.

Measurement of the Fluorescence Intensities of BC Cells in Co-Culture

Same protocol as for Example 2 “Measurement of the fluorescence intensities of BC and LC cells in co-culture” except that the image analysis process allows a simultaneous and precise quantification of each proliferation status (reported by the FUCCI reporter) and of the induction of apoptosis (reported by the zipGFP-Casp3 reporter) in BC cells expressing the FUCCI reporter and in BC cells expressing the zipGFP-Casp3 reporter, respectively.

Results:

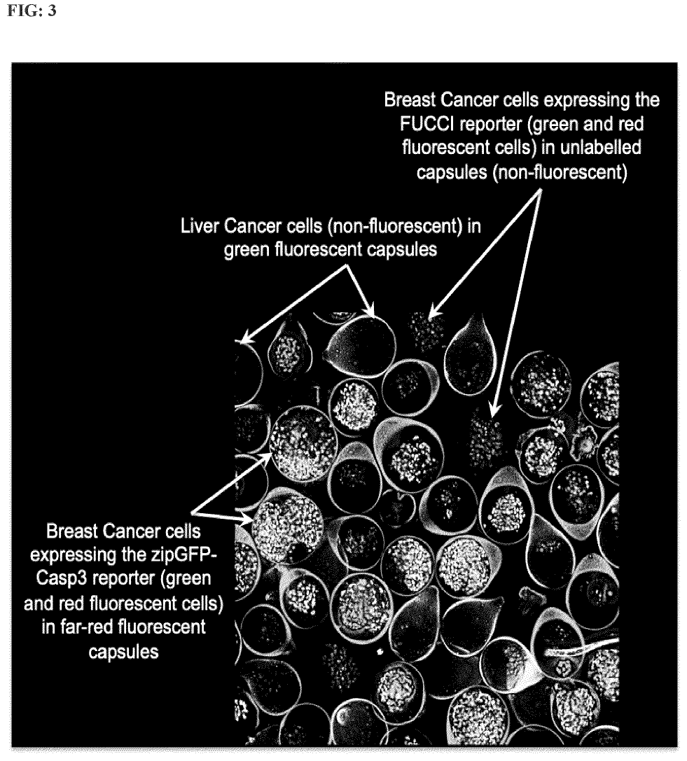

FIG. 3 shows a multi-culture of three different types of encapsulated cells: LC cells, BC cells stably expressing the reporter gene zipGFP-Casp3, and BC cells stably expressing the dual-reporter gene FUCCI. Capsule colors allows for the identification of each type of cells, and cell fluorescence intensities can be measured to quantify specific biological activities in each type of capsule (here proliferation of BC cells in unlabelled capsules or apoptosis of BC cells in far-red capsules). In this particular experiment, Applicants used different color of capsules to discriminate two different reporters having the same fluorescent colors, with an additional capsule color to identify a second cell type (LC cells).

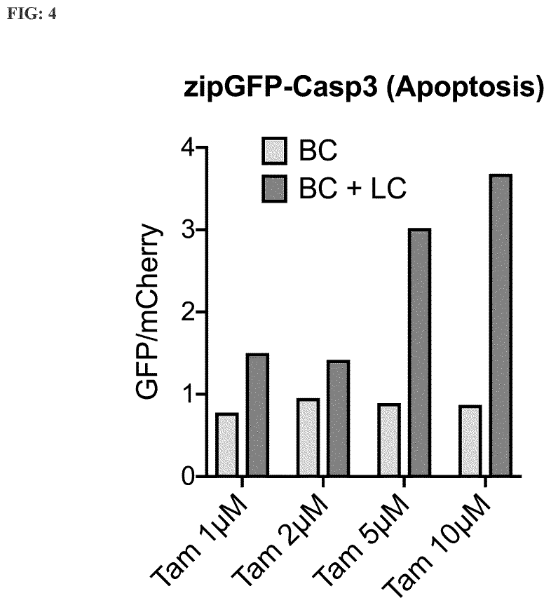

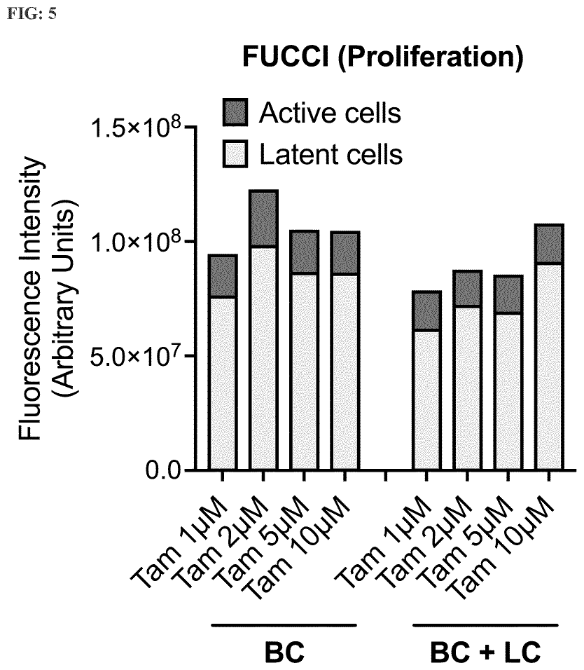

FIG. 4 and FIG. 5 show that Applicants can simultaneously measure two different biological activities in a multi-culture (co-culture) well with reporters for two different biological activities (cell proliferation and apoptosis). In this experiment, Applicants used micromolar range concentrations of Tamoxifen. At these concentrations of Tamoxifen, the inhibition of cell proliferation is saturated, but apoptosis is induced in a dose-dependent manner. Applicants can observe in FIG. 4 that the induction of apoptosis in BC cells is increased by Tamoxifen in a dose-dependent manner only in co-culture with LC cells. In FIG. 5, cell proliferation inhibition is already maximal at 1 μM and cannot be further increased with higher concentrations of Tamoxifen. This experiment shows that with the invention's method Applicants can independently and simultaneously measure two different biological activities in a multi-culture. Applicants showed again that the presence of LC cells increases the efficiency of Tamoxifen on BC cells. Importantly, this experiment allows the reliable discrimination of two different pharmacological effects (inhibition of cell proliferation and induction of apoptosis) of the same molecule in the same culture well.

1. An in vitro method for drug testing that is capable of analyzing multiple biological processes in one or more cell types, the method comprising:
a) expressing in each of said one or more cell types to be analyzed in the same in vitro drug testing assay, at least one fluorescent or bioluminescent reporter gene for each biological process to be analyzed;
b) encapsulating each of the said one or more cell types to be analyzed in the same in vitro drug testing assay with a biopolymer of alginate, wherein in the case there are more than one cell type, all, or all but one, of the respective capsules are labelled, each with a different type of fluorophore;
c) co-culturing all the encapsulated cell types to be analyzed of step b) in the same in vitro drug testing assay, optionally with one or more additional unlabeled cell types that are optionally encapsulated with labeled or unlabeled biopolymers of alginate;
d) optionally exposing said co-culture to one or more drugs;
e) measuring the activity of said multiple biological processes in each cell type of step c) to be analyzed in the same in vitro drug testing assay, by individually analyzing through optical imaging, before and/or after exposure to said optional one or more drugs to be tested, the fluorescence or bioluminescent intensities of each fluorescent or bioluminescent reporter gene,
wherein when more than one cell types are co-cultured, each cell type to be analyzed is identified through optical imaging on the basis of the type of fluorophore labelling associated with the alginate capsule containing said cell type to be analyzed.

2. The in vitro method for drug testing according to claim 1, wherein the multiple biological processes are selected from the list comprising: cell proliferation, inflammation, tumor growth or inhibition, drug toxicity, angiogenesis, immune activation, detoxification responses, hormone response, xenobiotic response, genotoxic stress response and apoptosis.

3. The in vitro method for drug testing according to claim 1, wherein said one or more cell types are selected for co-culturing based on anticipated or confirmed physiological interactions between said cell types, and/or based on anticipated or confirmed effects of one or more of said cell types on the one or more drugs to be tested.

4. The in vitro method for drug testing according to claim 1, wherein the in vitro method comprises step d) in which said co-culture is exposed to one or more drugs.

5. The in vitro method for drug testing according to claim 1, wherein the in vitro method further comprises step f) in which said co-culture is exposed to one or more fluorescent reagents to measure one or more additional biological processes.

6. The in vitro method for drug testing according to claim 1, wherein the optical imaging is performed by microscopy and/or luminometer.

7. The in vitro method for drug testing according to claim 1, wherein, said at least one or more cell types are selected from the group comprising cell lines or primary tissue cell subsets of: hepatocytes, tumor cells, brain cells, epithelial cells, endothelial cells, immune system cells, pluripotent stem cells, embryonic stem cells, lung cells, kidney cells, artery cells, bone cells, cartilage cells, muscle cells, pancreatic cells, intestinal cells, skin cells, fibroblasts, fungal cells or bacteria.

8. The in vitro method for drug testing according to claim 1, wherein, at least one of said one or more cell types to be analyzed in the in vitro drug testing assay or at least one of the optionally added unlabeled cell types of step c) are hepatocytes.

9. The in vitro method for drug testing according to claim 1, wherein, at least one of said one or more type of encapsulated cell derives from a patient cancer sample.

10. The in vitro method for drug testing according to claim 1, wherein, at least one of said one or more cell types is a bacterium or a fungal cell.

11. The in vitro method for drug testing according to claim 10, wherein the drug to be tested is an antibiotic or a fungicide.

12. The in vitro method for drug testing according to claim 10, wherein at least one of said multiple biological processes to be analyzed is selected from the list comprising: anti-microbial resistance or susceptibility, bacterial proliferation, bactericidal or bacteriostatic activity.

13. An in vitro drug testing kit suitable for testing the effect of one or more drugs of interest on multiple biological processes in one or more target cell types, the kit comprising:
at least one ready-to-use microwell plate containing one or more alginate-encapsulated target cell types expressing a fluorescent or bioluminescent reporter gene for each biological process to be analyzed in said one or more target cell types, wherein in case there are more than one cell type, all, or all but one, of the respective capsules are labelled, each with a different type of fluorophore.

14. The in vitro drug testing kit according to claim 13, wherein at least one of said one or more target cell types are hepatocytes.

15. The in vitro drug testing kit according to claim 13, wherein said fluorophore-labelled alginate-encapsulated one or more target cell types are co-cultured with one or more patient-derived cell types.

16. The in vitro drug testing kit according to claim 15, wherein the one or more patient-derived cell types are genetically engineered to contain at least one fluorescent reporter gene for each multiple biological process to be analyzed.

17. The in vitro drug testing kit according to claim 15, wherein the one or more patient-derived cell types are each encapsulated with a biopolymer of alginate labelled with a fluorophore.

18. The in vitro drug testing kit according to claim 13, wherein the multiple biological processes to be analyzed are selected from the list comprising: cell proliferation, inflammation, tumor growth or inhibition, drug toxicity, angiogenesis, immune activation, detoxification responses, hormone response, xenobiotic response, genotoxic stress response and apoptosis.

19. The in vitro drug testing kit according to claim 13, wherein the one or more target cell types are selected from the list comprising: hepatocytes, tumor cells, brain cells, epithelial cells, endothelial cells, immune system cells, pluripotent stem cells, embryonic stem cells, lung cells, kidney cells, artery cells, bone cells, cartilage cells, muscle cells, pancreatic cells, intestinal cells, fibroblasts or skin cells.

20. The in vitro testing kit according to claim 13, wherein said one or more drugs are tested per well of said at least one ready-to-use microwell plate to identify the effects of said one or more drugs on biological processes within said encapsulated target cell types.

21. The in vitro testing kit according to claim 13, wherein more than one drug are tested per well of said at least one ready-to-use microwell plate to identify interactions between said drugs.
